SEQUENCE LISTING (7 sequences)

SEQ ID NO: 1 - DNA, 539 bp, Homo sapiens
  misc_feature (239)..(239): n=g/c
cttacttcgagtctgttccttcccccagtcatgcctctgctgctgctgttaccagtccaaaagtctgatgacctcggtccccatgaacggaacaagcatccagtggaagaaccacgatcaaaacaaccacaacacagaccggagcagccataaggacagcatgaactgaccacccttagaagcactcctcggtactcccataatcctctcggagaaaaaaatcacaaggcaactgtgantccgggaatctcttctctgatccttcttccttaattcactcccacacccaagaagaaatgctttccaaaaccgcaagggtagactggtttatccacccacaacatctacgaatcgtacttctttaattgatctaatttacatattctgcgtgttgtattcagcactaaaaaatggtgggagctgggggagaatgaagactgttaaatgaaaccagaaggatatttactacttttgcatgaaaatagagctttcaagtacatggctagcttttatggcagttctggtgaatgttcaatggg

SEQ ID NO: 2 - DNA, 601 bp, Homo sapiens
  misc_feature (301)..(301): n=g/a
gggggcggcagctttgtgctttttagtggccgcgtcccaggatagctggaaggttaggacgctcttgcggtcccagagtggagtggaaggtctggagctttgggaggagacggggaggacagactggaggcgtgttcctccggagttttctttttcgtgcgagccctcgcgcgcgcgtacagtcatcccgctggtctgacgattgtggagaggcggtggagaggcttcatccatcccacccggtcgtcgccggggattggggtcccagcgagacctccccgggagaagcagtgcccaggangttttctgaagccggggaagctgtgcagccgaagccgccgccgcgccggagcccgggacaccggccaccctccgcgccacccaccctcgccggctccggcttcctctggcccaggcgccgcgcggacccggcagctgtctgcgcacgccgagctccacggtcggtgcaagtctttcttatcggggactgggactggggcgggtgcggggatggcggagacgctgcctgggcccctcggtcgggagaagacgagagctgggaacgttctggcccgaccgccctgcagcttgggcgacccgt

SEQ ID NO: 3 - DNA, 21 bp, Homo sapiens
tgaaaagttgtagtatgcccc

SEQ ID NO: 4 - DNA, 41 bp, Homo sapiens
catttaaaatactgaagctccacaatttggtgaattatcga

SEQ ID NO: 5 - DNA, 45 bp, Homo sapiens
tggttaaaaaaaaaggaatagcatcaatagtgagtttgttgtcct

SEQ ID NO: 6 - DNA, 24 bp, Homo sapiens
accctacaacagacccacacaata

SEQ ID NO: 7 - DNA, 601 bp, Homo sapiens
  misc_feature (301)..(301): n=a/t
gttaacatgcattagaaaactaaatcttttgaaaagttgtagtatgccccctaagagcagtaacagttcctagaaactctctaaaatgcttagaaaaagatttattttaaattacctccccaataaaatgattggctggcttatcttcaccatcatgatagcatctgtaattaactgaaaaaaaataattatgccattaaaagaaaatcatccatgatcttgttctaacacctgccactctagtactatatctgtcacatggtactatgataaagttatctagaaataaaaaagcatacanttgataattcaccaaattgtggagcttcagtattttaaatgtatattaaaattaaattattttaaagatcaaagaaaactttcgtcatactccgtatttgataaggaacaaataggaagtgtgatgactcaggtttgccctgaggggatgggccatcagttgcaaatcgtggaatttcctctgacataatgaaaagatgagggtgcataagttctctagtagggtgatgatataaaaagccaccggagcactccataaggcacaaactttcagagacagcagagcacacaagcttctagg

(19) United States
(12) Patent Application Publication
(10) Pub. No.: US 2009/0162855 A1
(43) Pub. Date: Jun. 25, 2009

(54) Identification of Genetic Variants Associated with Increased Severity of Pulmonary Disease

(75) Inventors: Mitchell Drumm, Brecksville, OH (US); Rebecca Darrah, Strongsville, OH (US); Paul Smith, Mayfield Village, OH (US); Steven Martin, Shawnee, KS (US); Michael Knowles, Chapel Hill, NC (US); Steven Strausbaugh, Novelty, OH (US); Robert Schilz, Shaker Heights, OH (US)

Correspondence Address: HAHN LOESER & PARKS, LLP, One GOJO Plaza, Suite 300, AKRON, OH, 44311-1076, US

(73) Assignee: CASE WESTERN RESERVE UNIVERSITY, Cleveland, OH (US)

(21) Appl. No.: 12/264,165

(22) Filed: Nov. 3, 2008

Related U.S. Application Data
(60) Provisional application No. 60/985,143, filed on Nov. 2, 2007.

(51) Int. Cl. C12Q 1/68

(52) U.S. Cl. 435/6

(57) ABSTRACT

A method of determining a genetic component contributing to the severity of a pulmonary disease in a patient comprises determining the presence or absence of one or more single nucleotide polymorphisms (SNPs) in the Endothelin Receptor A (EDNRA) gene or the Interleukin-8 (IL-8) gene of the patient. The SNPs are rs5335 or rs1801708 for EDNRA, or rs4O73 for IL-8. The pulmonary disease may be cystic fibrosis or lymphangioleimyomatosis. Determining the presence or absence of one or more of SNPs rs5335 or rs1801708 in the EDNRA gene or rs4073 in the IL-8 gene of the patient may also be used in a method of treating a patient having a pulmonary disease. A kit may comprise one or more probes for determining the presence or absence of one or more of SNPs rs5335 and rs1801708 in the EDNRA gene or the SNP rs4073 in the IL-8 gene.

### STATEMENT REGARDING FEDERALLY SPONSORED RESEARCH OR DEVELOPMENT

This invention was made with government support under National Institutes of Health grant numbers HL68890 and T32HL-07415. The U.S. government may have certain rights to the invention.

### BACKGROUND OF THE INVENTION

This invention relates to the identification of genetic factors that contribute to the severity of pulmonary diseases. More particularly, this invention relates to the identification of genetic factors that contribute to the severity of cystic fibrosis and lymphangioleimyomatosis (LAM). It is further envisioned that these genetic factors may also contribute to the severity of other diseases such as asthma, pulmonary hypertension, and systemic hypertension.

Cystic fibrosis (CF) is an obstructive airway disease, characterized by chronic infection and inflammation and caused by mutations in the cystic fibrosis transmembrane conductance regulator (CFTR) gene. Like many monogenic disorders, CF has a wide range in disease severity that can not be due only to the CFTR mutation. The presence of non-CFTR genetic polymorphisms may explain differences in phenotype among individuals with CF.

In CF, the inflamed tissue releases mediators that cause smooth muscle contraction and proliferation, causing the bronchoconstriction characteristic of CF and asthma. A therapeutic regimen for the CF airway often includes β₂-adrenergic receptor agonists to stimulate smooth muscle relaxation and bronchodilation. Steroids and non-steroidal anti-inflammatory agents are also used to suppress the inflammatory signals that cause smooth muscle contraction. These pharmacologic manipulations of airway mechanics are effective therapeutically for CF, but the potential to improve these treatments may be realized with better understanding of the pathways involved.

The influence of genetics on airway disease has been estimated to account for 50% or more of the phenotypic variance. Variants in the gene encoding transforming growth factor β₁ associate with CF lung function, but probably account for a relatively small amount of the phenotypic variance. Variants in the β₂-adrenergic receptor gene associate with differences in acute response to agonists similar to that reported for asthma, but no evidence was found that these same variants associate with long-term differences in lung function in CF patients.

Lymphangioleimyomatosis (LAM) is also a pulmonary disease but is characterized by tumors that derive from smooth muscle and are found largely in the lungs. These tumors tend to block the flow of air in the lungs. LAM is specific to females only

Endothelin-1 (EDN-1) is a pro-inflammatory molecule and has been found to be elevated in the sputum and plasma of CF patients. EDN1 is the ligand for Endothelin Receptor A (EDNRA). Interleukin-8 (IL-8) is also a component of CF lung disease; it is elevated, with substantial variation, in the lungs of CF patients

### SUMMARY OF THE INVENTION

It is, therefore, an aspect of the present invention to provide a method of determining a genetic component contributing to the severity of a pulmonary disease such as cystic fibrosis or lymphangioleimyomatosis.

It is another aspect of the present invention to provide a method of predicting a patient's response to treatments for cystic fibrosis, such as treatment with an EDNRA antagonist or an IL-8 antagonist. In one example, sitaxentan is the EDNRA antagonist.

A method of determining a genetic component contributing to the severity of a pulmonary disease in a patient comprises determining the presence or absence of one or more single nucleotide polymorphisms (SNPs) in the Endothelin Receptor A (EDNRA) gene or the Interleukin-8 (IL-8) gene of the patient. The SNPs are rs5335 or rs 1801708 for EDNRA, or rs 4073 for IL-8. The pulmonary disease may be cystic fibrosis or lymphangioleimyomatosis. Determining the presence or absence of one or more of SNPs rs5335 or rs1801708 in the EDNRA gene or rs 4073 in the IL-8 gene of the patient may also be used in a method of treating a patient having a pulmonary disease. In one example, the pulmonary disease is selected from the group consisting of cystic fibrosis and lymphangioleimyomatosis. In another example, the SNP rs5335 comprises a guanine (G) or a cytosine (C) nucleotide corresponding to position 239 of SEQ. ID. NO. 1. In another example, rs1801708 comprises a guanine (G) or adenine (A) nucleotide corresponding to position 301 of SEQ. ID. NO. 2. In still another example, rs 4073 comprises an adenine (A) or a thymidine (T) nucleotide corresponding to position 301 of SEQ. ID. NO. 7.

A method of treating a patient having a pulmonary disease, may comprise determining the presence or absence of one or more single nucleotide polymorphisms (SNPs) in the Endothelin Receptor A (EDNRA) gene or the Interleukin-8 (IL-8) gene of the patient, wherein the SNPs are selected from group consisting of rs5335 and rs1801708 for EDNRA and rs 4073 for IL-8. In one example, the pulmonary disease is selected from the group consisting of cystic fibrosis and lymphangioleimyomatosis. In another example, the SNP rs5335 comprises a guanine (G) or a cytosine (C) nucleotide corresponding to position 239 of SEQ. ID. NO. 1. In another example, rs1801708 comprises a guanine (G) or adenine (A) nucleotide corresponding to position 301 of SEQ. ID. NO. 2. In still another example, rs 4073 comprises an adenine (A) or a thymidine (T) nucleotide corresponding to position 301 of SEQ. ID. NO. 7.

A kit for determining the presence or absence of one or more single nucleotide polymorphisms (SNPs) in the Endothelin Receptor A (EDNRA) gene or the Interleukin-8 (IL-8) gene of the patient may comprise one or more probes for determining the presence or absence of one or more of SNPs rs5335 and rs1801708 in the EDNRA gene or the SNP rs 4073 in the IL-8 gene of the patient. In one example, wherein rs5335 comprises a guanine (G) or a cytosine (C) nucleotide corresponding to position 239 of SEQ. ID. NO. 1. In another example, rs1801708 comprises a guanine (G) or adenine (A) nucleotide corresponding to position 301 of SEQ. ID. NO. 2. In still another example, rs4073 comprises an adenine (A) or a thymidine (T) nucleotide corresponding to position 301 of SEQ. ID. NO. 7. In yet other examples, the probe is a nucleic acid comprising at least residues 221-239 of SEQ. ID. NO. 1, at least residues 239-250 of SEQ. ID. NO. 1, at least residues 290-301 of SEQ. ID. NO. 2, at least residues 301-320 of SEQ. ID. NO. 2, at least residues 283-301 of SEQ. ID. NO. 7, at least residues 301-319 of SEQ. ID. NO. 7, or the reverse complement thereof. In still other examples, a nucleic acid probe may additionally extend beyond the SNP by 1, 2, 3, 4, 5, 10 or more nucleic acids.

### BRIEF DESCRIPTION OF THE SEVERAL VIEWS OF THE DRAWINGS

FIG. 1A is a schematic representation of “blocks” of markers in the EDNRA gene showing linkage disequilibrium with pairwise correlation coefficients of r²>0.85;

FIG. 1B is a schematic representation of the exon/intron structure of the EDNRA gene;

FIG. 1C provides the relative positions of the SNPs tested in EDNRA gene;

FIG. 1D is a plot of the -Log10 of the p-values for each SNP's association with pulmonary phenotype in the multi-center U.S. population;

FIG. 1E is a plot of the -Log10 of the p-values for each SNP's association with pulmonary phenotype in the Seattle population;

FIG. 1F is a plot of the -Log10 of the p-values for each SNP's association with pulmonary phenotype in the Ireland population;

FIG. 2 is a pair of bar graphs of estimated forced expiratory volume in 1 second (FEV₁) according to genotype at rs 5335 for the multi-center cohort (A) and for a combined Ireland Seattle/Cleveland cohort;

FIG. 3A is a series of graphic representations and a standard curve generated by mixing C and G alleles of rs5335 in known proportions, amplifying by PCR and then measuring the fluorescence signal of each allele from a single base extension sequencing reaction;

FIG. 3B is a graph showing % C allele expressed for genomic and mRNA;

FIG. 3C is a graph of relative EDNRA mRNA copy number for the three genotypes of rs 5335;

FIG. 4 is a panel of bar graphs showing the average estimated pulmonary function of cystic fibrosis patients having certain SNP alleles;

FIG. 5 is a panel of bar graphs showing the relative copy number of EDNRA mRNA for cystic fibrosis patients having certain SNP alleles for rs1801708 (Panel A), rs5335 (Panel B) and a combination of rs1801708 and rs5335 (Panel C);

FIG. 6 is a schematic representation of the EDNRA gene;

FIG. 7 is a bar graph providing the relative EDNRA mRNA copy number for smooth muscle cell lines from males and females, with and without estrogen stimulation;

FIG. 8 is a pair of graphs showing the effects of mitogen treatments on ASM proliferation;

FIG. 9 is a bar graph showing the effects of estrogen on smooth muscle cell proliferation;

FIG. 10 is a micrograph of cells stained using indirect immunocytochemistry for EDRNA;

FIG. 11 is a pair of micrographs of cultured ASM cells placed in either serum free media (A) or media containing ET-1 (B) for 24 hr; and

FIG. 12 is a graph showing the response of cultures of smooth muscle cells to endothelin-1 (ET-1) acutely (control) and sarcoplasmic reticulum release of calcium measured by Fura-2 fluorescence ratios.

### DETAILED DESCRIPTION OF THE INVENTION

A method of determining a genetic component contributing to the severity of cystic fibrosis or lymphangioleiomyomatosis is provided. It is envisioned that the same method is applicable to determining a genetic component contributing to the severity of smooth muscle proliferative diseases, such as asthma, pulmonary hypertension, and systemic hypertension.

A method of predicting a patient's response to treatments for cystic fibrosis or lymphangioleiomyomatosis, such as treatment with EDNRA antagonists or IL-8 antagonists, is also provided. With the knowledge of a patient's status regarding EDNRA and IL-8 genotypes, treatment for pulmonary diseases such as cystic fibrosis may be adjusted. For example, in those cases where a patient having CF accompanied by a “severe” genotype with regard to EDNRA, more aggressive prophylaxis may be prescribed. Similarly, treatment options may also be optimized with regard to other smooth muscle proliferative diseases upon determining a patient's genotype with regard to EDNRA.

Airway inflammation and pulmonary disease are heterogeneous phenotypes in cystic fibrosis (CF) patients, even among patients with the same CFTR genotype. Endothelin, which is involved in inflammatory response and smooth muscle signaling, is increased in CF airways, potentially contributing to the pulmonary phenotype. Four cohorts of cystic fibrosis patients were screened for variants in endothelin pathway genes to determine if any of these variants associated with pulmonary function. An initial cohort of 808 patients ascertained from across the United States and homozygous for the common CF mutation, ΔF508, showed significant association for two polymorphisms near EDNRA. Variants within EDNRA were examined in three additional cohorts of CF patients: 238 patients from Seattle, Wash.; 305 CF patients from Dublin, Ireland; and 228 patients from Cleveland, Ohio, for a total of 1,579 CF patients. All four of the groups tested showed the same association between EDNRA variants and pulmonary function. Single nucleotide polymorphism rs5335, located in the 3′UTR, displayed p=0.04, 0.003, 0.009, and 0.04 for the 4 groups tested, respectively. At the molecular level, both quantitative PCR and single nucleotide primer extension assays have confirmed that mRNA from cultured, primary non-CF smooth muscle cells carrying the allele associating with poor pulmonary function expresses higher mRNA levels than the allele associated with better lung function.

Thirteen polymorphisms encompassing 5 genes, those encoding CD14, EDNRA, interleukins 3, 4 and 9 (IL3, IL4, and IL9) were tested and associated (P<0.05) with pulmonary disease in the initial cohort of 808 CF patients homozygous for the ΔF508 mutation and classified as having either severe or mild lung disease. Each single nucleotide polymorphism (SNP) was examined to determine the best fitting genetic model and whether 2-locus haplotypes showed more significant associations than individual SNPs. CD14 and IL9 alleles exhibit a dominant inheritance pattern and relative risks (rr) for being severe of 1.6-1.8 for CD14, 1.3-1.4 for IL9. IL3 and IL4 alleles display an additive inheritance pattern (rr=2.1-2.2 for IL3, 5.0 for IL4). No clear gender effects were observed for CD14, IL3, IL4 or IL9 markers. Table 1 provides a listing of genes with at least one marker associating with pulmonary severity.

TABLE 1
Markers associating with lung disease in 808 ΔF508
homozygous CF patients

| N | rs Number | Gene | Chr. | BP | P |
|---|---|---|---|---|---|
| 728 | rs2569190 | CD14 | 5 | 139993100 | 0.043 |
| 804 | rs2569191 | CD14 | 5 | 139994087 | 0.030 |
| 733 | rs753279 | CD14 | 5 | 140004002 | 0.013 |
| 742 | rs1801708 | EDNRA | 4 | 148759974 | 0.045 |
| 710 | rs5335 | EDNRA | 4 | 148821445 | 0.014 |
| 727 | rs5342 | EDNRA | 4 | 148822376 | 0.042 |
| 804 | rs1517137 | EDNRA | 4 | 148835482 | 0.033 |
| 731 | rs31400 | IL3 | 5 | 131417406 | 0.036 |
| 728 | rs27348 | IL3 | 5 | 131435038 | 0.012 |
| 804 | rs246351 | IL3 | 5 | 131537170 | 0.012 |
| 732 | rs1468216 | IL4 | 5 | 132064151 | 0.042 |
| 723 | rs2304075 | IL9 | 5 | 135206023 | 0.034 |
| 729 | rs28792 | IL9 | 5 | 135299261 | 0.019 |

Patients with severe lung disease were compared to all mild groups combined 116 markers in 18 genes were tested, 13 markers and 5 genes were significant using P < 0.05.

As part of a larger screen for genetic variants that modify CF lung disease, candidate genes were grouped into functional classes. Table 2 shows a panel of variants grouped both for their role in smooth muscle contraction and relaxation in a CF context, and their role in endothelin signaling.

TABLE 2
List of polymorphisms examined in the initial genetic screen

| Gene | Protein | reference SNP # |
|---|---|---|
| EDN1 | Endothelin 1 | rs5370 |
| | | rs500843 |
| | | rs5369 |
| | | rs17765653 |
| EDN2 | Endothelin 2 | rs915025 |
| | | rs3768274 |
| EDN3 | Endothelin 3 | rs260743 |
| | | rs154872 |
| | | rs107421 |
| EDNRA | Endothelin receptor A | rs3756022 |
| | | rs5333 |
| | | rs5335 |
| | | rs1517137 |
| | | rs1517131 |
| | | rs1801708 |
| | | rs5343 |
| EDNRB | Endothelin receptor B | rs1924919 |
| NOS1 | Nitric oxide synthase 1 | rs2682826 |
| NOS2A | Nitric oxide synthase 2 | rs1137933 |
| | | rs367455 |
| | | rs944722 |
| | | rs3794764 |
| | | rs3730013 |
| | | rs2297518 |
| NOS3 | Nitric oxide synthase 3 | rs1799983 |
| | | rs1800779 |
| | | rs1799983 |
| | | rs743507 |
| | | rs3730011 |

As an initial screen, each of the variants was tested for association with pulmonary function in the initial cohort of 808 patients by determining if genotype frequencies differed between patients with lung function in the upper quartile for their birth cohort and those with lung function in the lower quartile. SNPs in or near EDNRA appeared significant (p<0.05, Table 3).

TABLE 3
EDNRA SNPs tested in original cohort demonstrating
a significant level of discordance

| Gene | Reference SNP # | p (mild vs. severe) |
|---|---|---|
| EDNRA | rs5335 | 0.01403 |
| EDNRA | rs1517137 | 0.03285 |
| EDNRA | rs1801708 | 0.04479 |

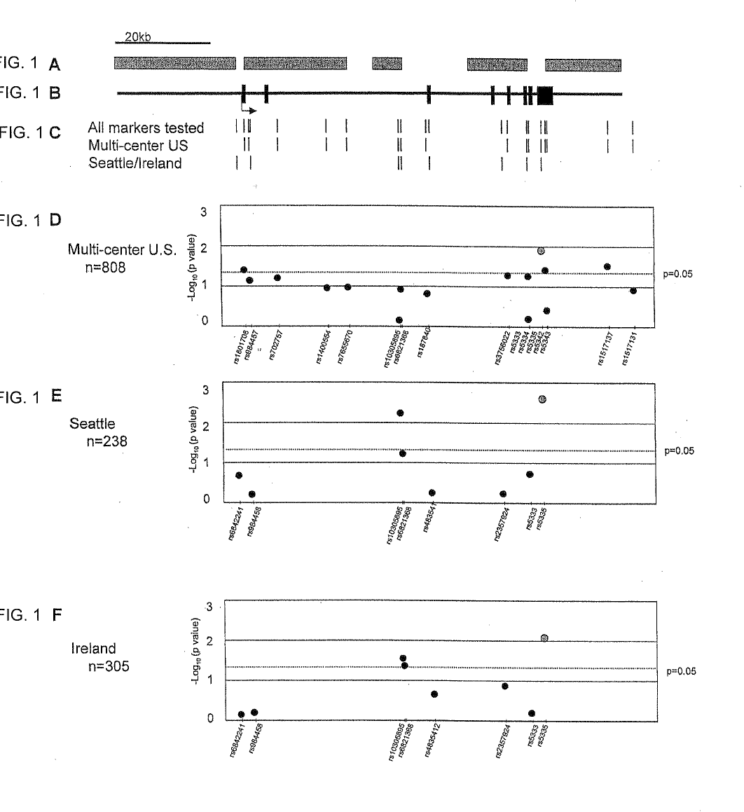

Additional EDNRA SNPs within and around EDNRA were genotyped, for a total of 15 SNPs (FIG. 1D-1F). Four of the 15 SNPs showed a significant (p<0.05) association with phenotypic classification, and four were not all in significant linkage disequilibrium with each other.

Two additional groups of CF patients were genotyped for variants spanning the EDNRA genomic region: one from Seattle, Wash. and one from Dublin, Ireland. As shown in FIGS. 1D-1F, both of these cohorts show similar results compared to the multi-center U.S. cohort, in that variants within EDNRA associate with CF pulmonary function. SNP rs5335 showed concordance in all three groups, so the fourth cohort of CF patients from Cleveland, Ohio was genotyped for this variant (p=0.04). Details regarding the test cohorts can be found in Table 4.

TABLE 4
Allele and Genotype frequencies of rs5335 in 4 patient cohorts

| | C | G | CC | CG | GG |
|---|---|---|---|---|---|
| U.S. (808) | 0.405 | 0.595 | 0.141 | 0.525 | 0.334 |
| Cleveland (228) | 0.390 | 0.610 | 0.169 | 0.441 | 0.390 |

FIG. 1 is a schematic representation of the EDNRA gene and position of polymorphisms analyzed. In FIG. 1A, the grey boxes indicate “blocks” of markers showing linkage disequilibrium generated by Haploview with pairwise correlation coefficients of r²>0.85. FIG. 1B is a schematic representation of the exon/intron structure of the EDNRA gene (the arrow indicates the start and direction of transcription) and FIG. 1C provides the position of the SNPs tested in EDNRA gene. The top set of lines show the composite of all markers tested, the middle set of lines are those tested in the multi-center cohort and the bottom set of lines are SNPs genotyped in the Seattle and Ireland cohorts. Plots of the -Log10 of the p-values for each SNPs association with pulmonary phenotype in the multi-center U.S., Seattle and Ireland populations are provided as FIGS. 1D, 1E and 1F, respectively. At the bottom of FIGS. 1D-1F, SNPs are indicated by their respective reference SNP reference sequence (rs) numbers.

The original test cohort of 808 CF patients (Multi-center U.S.) were analyzed by χ² test of association for association with pulmonary phenotype and of the 15 SNPs tested within and around EDNRA, 4 SNPs displayed significance at p<0.05: rs1801708, rs5335, rs5342, and rs1517137 (FIG. 1D). Multiple associating SNPs in the same gene may be due to a lack of independence (linkage disequilibrium) between markers. To address this possibility, these 4 SNPs were assessed to determine the degree of disequilibrium exhibited between marker pairs (Table 5). Modest disequilibrium was apparent between the 3′ SNPs, rs5335, rs5342 and rs1517137 (0.54<r²<0.75), but rs1801708 showed no evidence of disequilibrium with any of the other SNPs (r²≦0.035). These results are consistent with the LD map, as shown in FIG. 1A.

TABLE 5
Correlation coefficients for significantly associating SNPs.
R²

| | | | | |
|---|---|---|---|---|
| rs1801708 | 1.000 | | | |
| rs5335 | 0.032 | 1.000 | | |
| rs5342 | 0.035 | 0.750 | 1.000 | |
| rs1517137 | 0.002 | 0.540 | 0.669 | 1.000 |
| | rs1801708 | rs5335 | rs5342 | rs1517137 |

Although the level of significance of the associations for individual markers was modest, linkage disequilibrium indicated at least two genetically distinct regions of the gene are involved. As it is unlikely that two regions of the same gene would show spurious associations in the same study, the EDNRA gene was pursued further and replication was chosen as a strategy for verification. Two additional, independently ascertained cohorts of CF subjects, from the Seattle, Wash. area (n=238) and Dublin, Ireland (n=305) were genotyped using 8 tagged SNPs representing the 5 linkage blocks surveyed in the U.S. multi-center cohort. Unlike the initial test cohort, these populations were not stratified by phenotype, but instead represented the entire range of pulmonary function. SNPs in intron 2 and the 3′ regions showed significant associations (FIG. 1), with the 3′ SNP rs5335 showing concordance between all 3 groups.

The 3′UTR SNP rs5335 demonstrated a strong association with pulmonary phenotype in each of the three independent studies. The final cohort analyzed was a group of CF patients from the Cleveland, Ohio area who, like the Seattle/Ireland cohort, were not stratified by pulmonary phenotype. This cohort was genotyped for rs5335 and, like the Seattle/Ireland cohort, showed a significant association between the rs5335 genotype and pulmonary function (p=0.04).

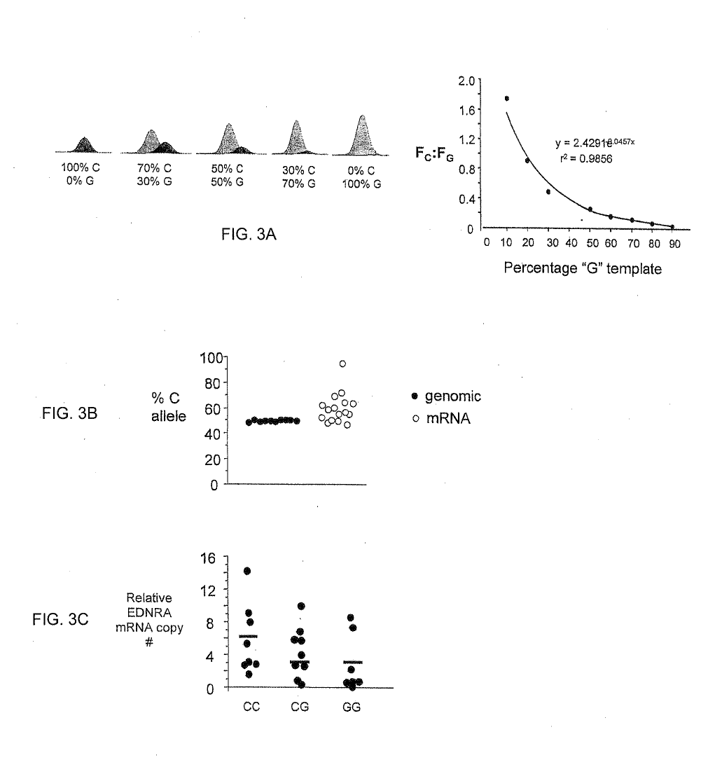

SNP rs5335 resides in the 3′ untranslated region of exon 8. The sequence of this SNP and surrounding nucleotides are provided as SEQ. ID. NO. 1. SNP rs1801708 is in the 5′ untranslated region (−231 bp from ATG). The sequence of this SNP and surrounding nucleotides are provided as SEQ. ID. NO. 2. Variants to either side in the coding region or 3′ to the gene and located only a few kb away, show weaker association with pulmonary function. The position of rs5335 suggests a regulatory role in mRNA expression. mRNA levels from cells of each genotype for rs5335 were compared. Primary smooth muscle cell cultures from non-CF tracheas were genotyped and mRNA levels of EDNRA were compared by two methods (FIG. 3). First, a single nucleotide primer-extension assay was utilized to compare relative expression of the C allele to the G allele at rs5335 in heterozygous cell lines. We were able to quantify this relative expression by using a standard curve generated by mixing known amounts of homozygous DNA. In cells heterozygous at rs5335, the C-containing allele was expressed at a level 20% greater on average than the G allele (FIGS. 3A and 3B). The proportion of G allele expressed never exceeded the C allele in any of the cell lines tested. Second, traditional quantitative PCR was used to examine the total amount of EDNRA mRNA present in the non-CF primary airway smooth muscle cells. When stratified by genotype, cells carrying the CC genotype expressed approximately three times the amount of EDNRA mRNA compared to cells homozygous for the GG genotype at rs5335 (p=0.03) (FIG. 3C).

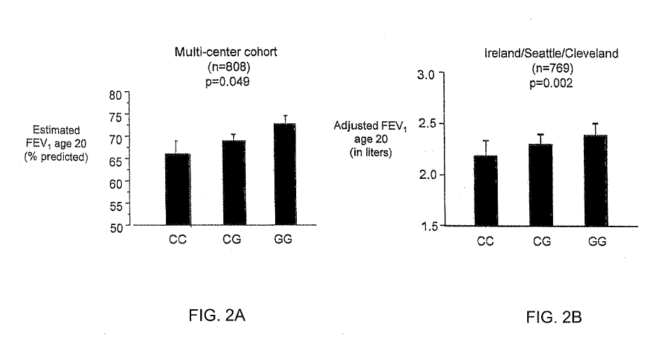

To summarize the genetic studies, all four independent CF populations show that homozygosity for the C allele at rs5335 is associated with significantly worse pulmonary function when compared those patients homozygous for the G allele (FIG. 2). In all of the populations tested, the CF patients homozygous for the C allele at rs5335 had pulmonary function levels interpolated or extrapolated for age 20 that were approximately 10% lower than those patients who were homozygous for the G allele at rs5335. Although these differences may seem subtle, a 10% change in pulmonary function can have significant clinical implications. The heterozygotes were intermediate and not statistically different than either homozygous group.

It is well recognized that males and females with CF are unequally affected. Females are known to have a reduced median survival age (by approximately 3 years) compared to males, as well as an earlier average age of Pseudomonas infection in the lungs, and greater rates of pulmonary decline. The association between alleles of the EDNRA gene and lung function was examined in males and females separately using the first cohort of 808 CF patients. As Table 6 demonstrates, sex of the subjects dramatically affects the association between alleles of the EDNRA gene and lung function. Twelve markers showed a significant association in females (0.044>p>0.00029), while only two markers were significant in males (0.021>p>0.013).

TABLE 6
EDNRA SNPs analyzed by gender

| | | | |
|---|---|---|---|
| | | χ² P | Genomic Control P |
| Gene | Reference SNP # | (mild vs. severe) | (mild vs. severe) |

Males

| | | | |
|---|---|---|---|
| EDNRA | rs3756022 | 0.013 | 0.0065 |
| EDNRA | rs5334 | 0.021 | 0.0097 |

Females

| | | | |
|---|---|---|---|
| EDNRA | rs1801708 | 0.00029 | 0.000068 |
| EDNRA | rs5335 | 0.00042 | 0.00035 |
| EDNRA | rs702757 | 0.00054 | 0.00012 |
| EDNRA | rs5342 | 0.00068 | 0.00073 |
| EDNRA | rs984457 | 0.0015 | 0.00036 |
| EDNRA | rs7655670 | 0.0016 | 0.00033 |
| EDNRA | rs1400554 | 0.0017 | 0.00036 |
| EDNRA | rs1517137 | 0.0042 | 0.0027 |
| EDNRA | rs6821368 | 0.012 | 0.0028 |
| EDNRA | rs1517131 | 0.018 | 0.016 |
| EDNRA | rs1878404 | 0.036 | 0.019 |
| EDNRA | rs5343 | 0.044 | 0.045 |

Similar levels of association are expected of markers exhibiting high linkage disequilibrium, and the markers in the 5′ block all significantly associate. As shown in Table 7, the two markers with the most significant associations, SNPs rs1801708 and rs5335, (p=0.00029 and p=0.00042, respectively, comparing severe and mild females) are located in the 5′ and 3′ untranslated regions (UTRs) and do not display linkage disequilibrium (LD) with each other (r²=0.0362). The significant associations of these two markers are therefore not due to LD.

TABLE 7
SNPs representing the 5′UTR and 3′UTR of EDNRA

| | | | | Frequency | Frequency in | |
|---|---|---|---|---|---|---|
| | Severe | Mild | | in mild | severe | P |
| SNP # | genotype | genotype | Position in gene | females | females | (mild vs. severe) |
| rs1801708 | AA | GG | 5′UTR | AA: 8.7% | AA: 17.7% | 0.00029 |
| | | | (−231 bp from ATG) | AG: 35.7% | AG: 41.4% | |
| | | | | GG: 55.6% | GG: 40.9% | |
| rs5335 | CC | GG | 3′UTR | CC: 13.5% | CC: 20.4% | 0.00042 |
| | | | (exon 8) | CG: 45.6% | CG: 60.1% | |
| | | | | GG: 40.9% | GG: 19.5% | |

Multiple significantly associating markers not in LD with each other in the general population is consistent with two mechanisms. One is that each associating region is distinct, each containing a variant affecting gene or protein function and the other is that these variants mark an underlying haplotype on which a functional variant resides. In either case, the two loci may demonstrate a cumulative, or additive effect. To assess this, the EM algorithm was first used to estimate haplotype frequencies in order to determine if two-locus haplotypes showed evidence of more significant association than individual markers. Haplotypes were analyzed using marker rs1801708 as the most significant marker representing the 5′ block, and marker rs5335 found in the 3′ region (Table 8). Overall, this combination showed a significant association (p=0.004), but when compared by gender only females showed a significant association (p=0.0000016 vs. p=0.54 for males).

TABLE 8
Two point haplotype associations of markers located
in the 5′UTR and 3′UTR EDNRA

| Gene | 5′SNP | 3′SNP | P value | Gender |
|---|---|---|---|---|
| EDNRA | rs1801708 | rs5335 | 0.547472 | Male |
| EDNRA | rs1801708 | rs5335 | 0.000002 | Female |
| EDNRA | rs1801708 | rs5335 | 0.004294 | Both |

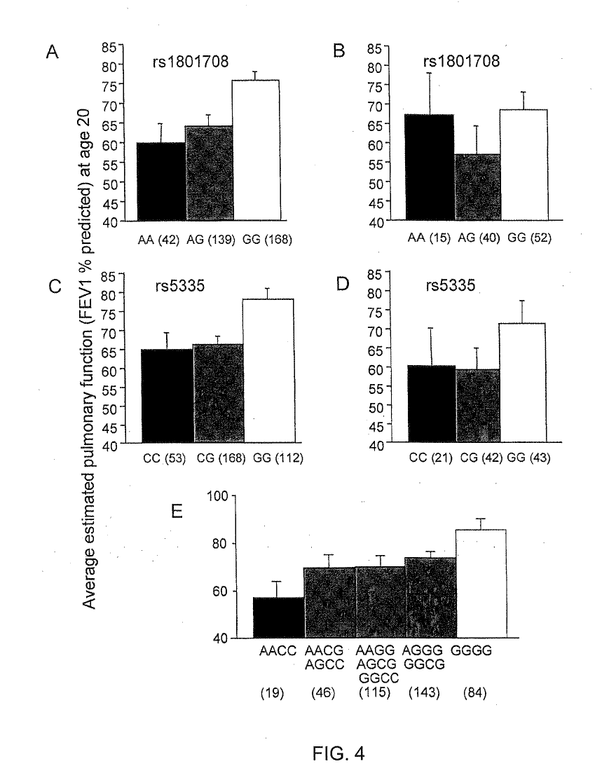

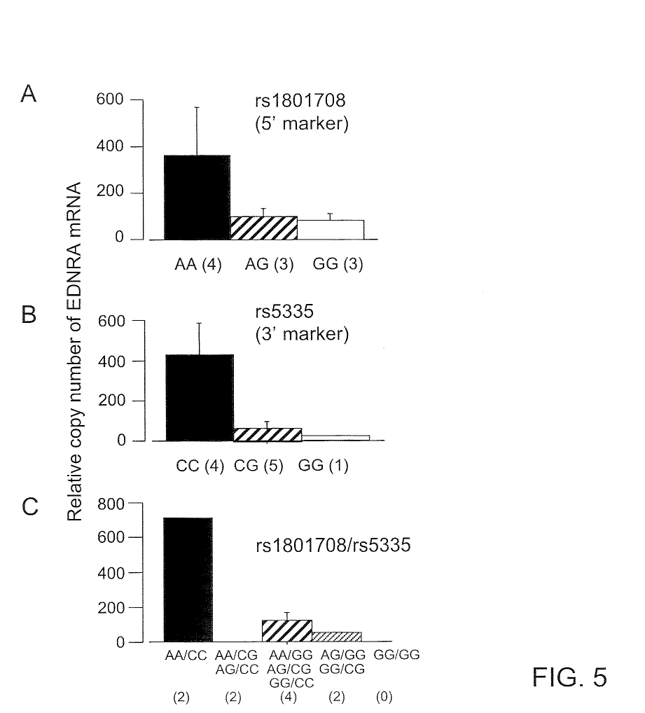

To estimate the magnitude of effects, we examined a set of 87 female patients not stratified for pulmonary function status and who were either homozygous for ΔF508, or carried one ΔF508 allele, and one allele with a nonsense mutation. These subjects were characterized using the same model for pulmonary function as the initial cohort. In FIG. 4, data for the original cohort are in panels A and C and data for the additional set of 87 females are in panels B and D. In the initial cohort of subjects, representing upper and lower quartiles of lung function, homozygosity for the G allele at rs1801708 or rs5335 confers a 15% and 13% greater estimated FEV₁ at age 20, respectively, when compared to those females homozygous for the A or C alleles (panels A and C). The data show that in the second cohort, rs5335 exhibits the same trend as was seen in the first cohort, as do the heterozygotes and GG homozygotes of rs1801708, although the AA homozygotes of this marker (panel B) do not.

Designating the G alleles at each locus as “mild” and the other as “severe”, the data from females of both cohorts were combined to compare the effects of both loci, rs 1801708 and rs5335 on pulmonary function estimates. FIG. 4E is a panel of bar graphs showing the average estimated pulmonary function of pooled cystic fibrosis patients from both groups was compared according to the number of “mild” alleles at rs1801708 and rs5335 combined (panel E). Those lacking mild alleles (the combined genotype of AA at rs1801708 and CC at rs5335) are designated AACC and those with 4 mild alleles are designated GGGG. The CF females with 0 mild alleles had an average estimated pulmonary function at age 20 of 56.5% (n=19), compared to 84.9% (n=84) for those CF females with 4 mild alleles (p=0.0017).

As shown in FIG. 4 E, subjects were classified by the number of mild alleles (0 to 4). While the single locus comparisons showed a difference of about 15% between homozygous groups of single markers, homozygotes for both loci differed by nearly 30% (56% vs 85%, p=0.0017).

Pulmonary function is tracked clinically as a marker of health in CF patients. Patients were examined by age group to determine if the genotypic proportions changed with age, possibly reflecting survival effects. There were 10 of 13 (77%) females homozygous for the severe alleles at both rs1801708 and rs5335 who were in the lower quartile for pulmonary function, leaving 3 (13%) in the upper quartile. Of the 146 females analyzed who are homozygous for ΔF508 and have reached the age of 29 (upper quartile for survival), none were homozygous for the severe allele at both rs1801708 and rs5335. Conversely, there were 61 females who were homozygous for the mild allele at each position. Of those 61 females, only 10 (16%) were in the lower quartile for pulmonary function, whereas 51 (84%) were in either the upper quartile for pulmonary function or survival (Table 9). Accordingly, haplotype comparison of the severe to younger mild vs severe to over 29 years of age generated p=0.0004 and 0.00007, respectively.

TABLE 9
Phenotypic breakdown of females grouped by
rs1801708/rs5335 combined genotypes

| | Upper | Upper | Lower |
|---|---|---|---|
| Genotype | Quartile | Quartile | Quartile |
| rs1801708/rs5335 | FEV₁ | Survival | FEV₁ |
| AA/CC (0 mild alleles) | 3 (13%) | 0 (0%) | 10 (77%) |
| n = 13 females | | | |
| GG/GG (4 mild alleles) | 31 (51%) | 20 (33%) | 10 (16%) |
| n = 61 females | | | |

As mentioned above, the markers demonstrating the most significant associations with CF pulmonary phenotype lie in putative regulatory regions of the gene (as opposed to the coding region). That is, the 5′ variants that show the most significant association lie in the 5′UTR and upstream of the first exon, consistent with transcriptional regulatory sequences. The 3′ variants show no association, except in the 3′ UTR of exon 8. A likely functional effect of 3′ UTR variants would be mRNA turnover.

Effects of these variants on EDNRA mRNA levels were examined. Primary airway smooth muscle cells from 10 non-CF individuals were harvested for DNA and RNA. Airway smooth muscle cells were cultured from tracheas obtained from autopsy. Cells were isolated from human trachealis muscle by digesting with collagenase and elastase in the presence of soybean trypsin inhibitor at 37° C. as previously described. The cells were harvested by centrifugation after filtering through Nytex and grown in Dulbecco's modified eagle's medium (DMEM/F12 1:1) containing 10% fetal bovine serum, 100 U/ml penicillin and 100 μg/ml streptomycin at 37° C. in a humidified atmosphere with 5% CO₂ and 95% air. DNA samples were genotyped for rs1801708 and rs5335 and EDNRA mRNA quantified from total RNA by real time RT-PCR. EDNRA mRNA was quantified relative to 18s rRNA in 10 airway smooth muscle cell lines and compared by genotype. RNA was isolated from airway smooth muscle cells using the Qiagen RNeasy kit. 1 μg of RNA was then reverse transcribed using Invitrogen M-MLV First Strand Synthesis. The resulting cDNA was then diluted 10 fold, and 2% of that was used in the following 25 μl PCR reaction: 1.5 mM MgCL₂, 1× buffer (Invitrogen), 0.25 mM dNTPs, 1U Platinum Taq (Invitrogen), 0.2 mM 5′primer (5′CATGCCTCTGCTGCTGCTGTTA3′), 0.2 mM 3′primer (5′AACCAGTCTACCTTGCGG3′) which generated a 297 bp fragment. SybrGreen was added to the reaction and amplification carried out on an MJ Scientific Opticon. A Cₜ was established for each sample and compared to standard curves to determine mRNA quantity. As an internal control for RNA quality and amount, a duplicate reaction was carried out using primers to amplify 18s rRNA. Values are expressed as a ratio of EDNRA: 18s copies. Only 2 lines were homozygous at both positions for the A and C alleles, but these two lines expressed nearly 8 times more EDNRA mRNA than any of the other genotypes. None of the lines obtained thus far are homozygous for G at both positions. Samples were compared by the same genotype criteria as shown in FIG. 4, by single SNP and by combination, but classified by the number of “mild” alleles. FIG. 5 shows that cells homozygous for the severe alleles (A at rs1801708 or C at rs5335) express 3 to 8 times more EDNRA mRNA than the other genotypes, and this is attributed mostly to those samples doubly homozygous for the two alleles.

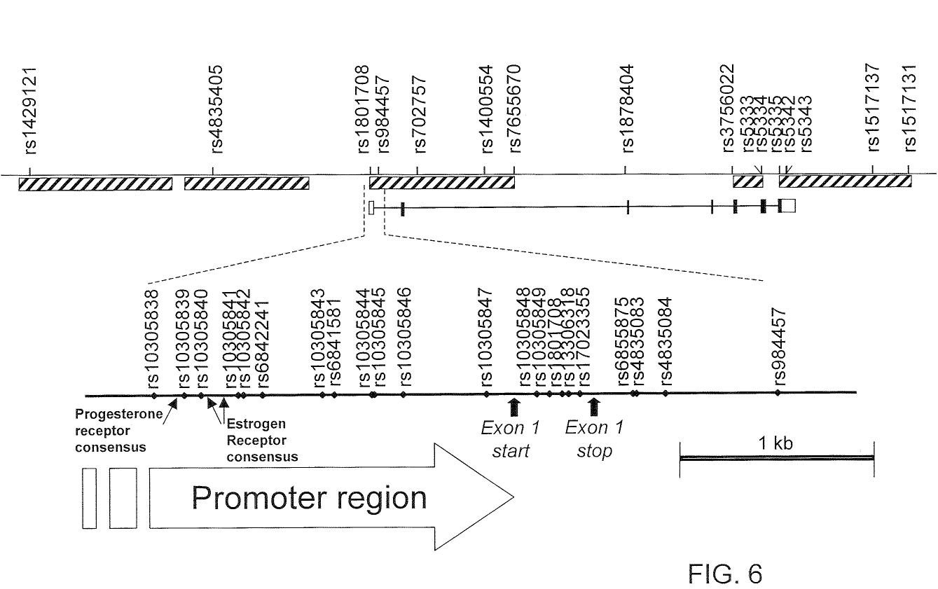

Because of the location of associating markers, the sequences encompassing the putative promoter region were scanned for potential regulatory elements. Three such elements were identified, as designated in FIG. 6. In FIG. 6, the putative promoter (large arrow) contains sequences matching estrogen and progesterone receptor binding sites. This interval also contains numerous sequence variants. We next examined this region to identify potentially functional polymorphisms in transcription factor binding motifs. As annotated in dbSNP (http://www.ncbi.nlm.nih.gov), this region is laden with variants, shown in the lower panel of FIG. 6.

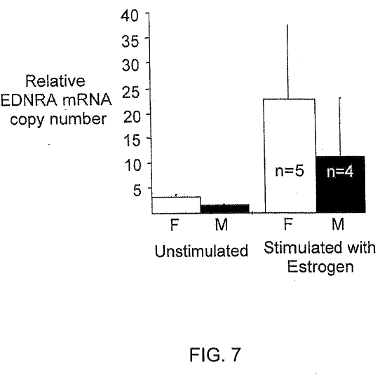

Sequence analysis suggested that gene expression of EDNRA should be responsive to estrogen. Nine smooth muscle cell lines were exposed to estrogen and mRNA levels compared to unstimulated cells. Five cell lines from females and 4 from males were treated with estrogen for 24 hours and then harvested for RNA. Quantitative RT-PCR was carried out to determine copy number using 18S rRNA as an internal control. As FIG. 7 shows, mRNA levels increased approximately 5-fold in response to hormone treatment. Both male and female cells responded similarly, although absolute levels were lower in male-derived cells.

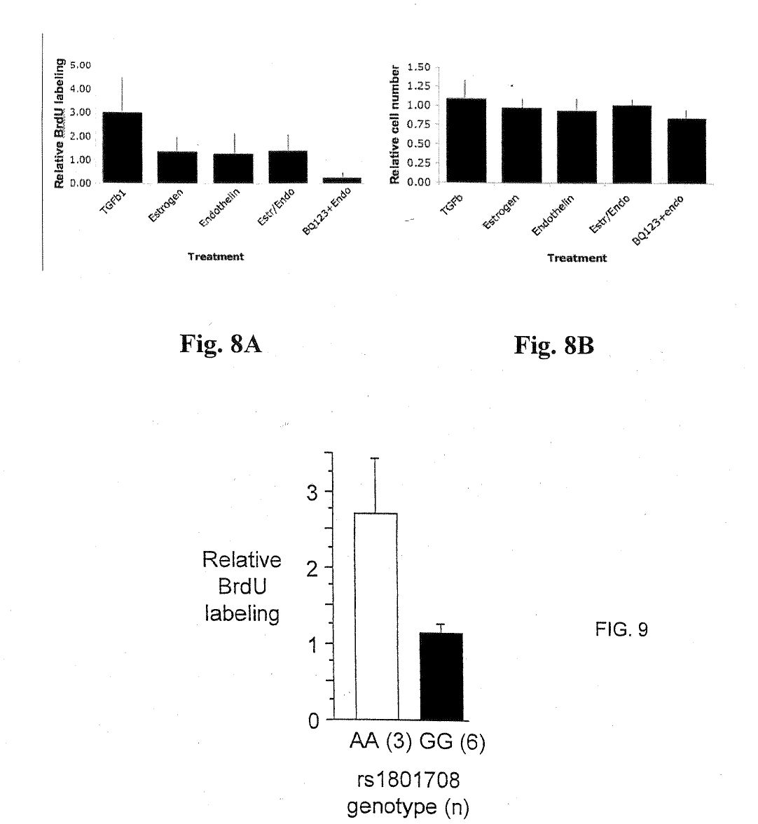

A cellular phenotype that could potentially explain the genotype-phenotype association is a difference in smooth muscle proliferation. To be able to assess this in vitro, we have carried out studies examining the mitogenic effects of the receptor agonist, endothelin, on cultured smooth muscle cells, as well as those of estrogen and Transforming Growth Factor Beta 1 (TGFβ1). In these studies, TGFβ1 is used as a control mitogen in that it exerts its effect on proliferation independent of the endothelin receptor. FIG. 8 shows complementary assays of cell growth and proliferation. Primary cultures of tracheal smooth muscle cells from 4 individuals were treated with TGFβ1, estrogen, endothelin, or estrogen and endothelin (Estr/Endo) for 72 hrs, and assayed for mitogenic index by BrdU incorporation (FIG. 8A) or for cell number (FIG. 8B). Values shown are ratios of treated to untreated (control) cells grown simultaneously. Each cell line and condition was carried out in triplicate. Whereas TGFβ1 tripled the mitogenic index of the cultures (FIG. 8A), it did not significantly alter cell number (FIG. 8B), indicating only a small number of cells divide in these cultures. An EDNRA antagonist, BQ123 (Sigma), was included at 1 μM to determine if EDNRA-mediated effects could be blocked. This maneuver had no effect on cell number (indicating it is not toxic), but reduced the mitogenic index of cells.

A larger group of cell lines stratified by genotype was examined. Primary cultures of tracheal smooth muscle cells from 9 individuals were treated with estrogen for 72 hrs, assayed for BrdU incorporation and compared to control cultures (no estrogen). FIG. 9 provides the ratio of treated to untreated cells. Estrogen appears to increase proliferation in cells harboring the “severe” AA genotype, but has no effect on cells with the “protective” GG genotype. Treatment with estrogen appears to result in increased proliferation, but the effect is genotype dependent. This is in contrast to other reports of vascular smooth muscle in which estrogen inhibits proliferation.

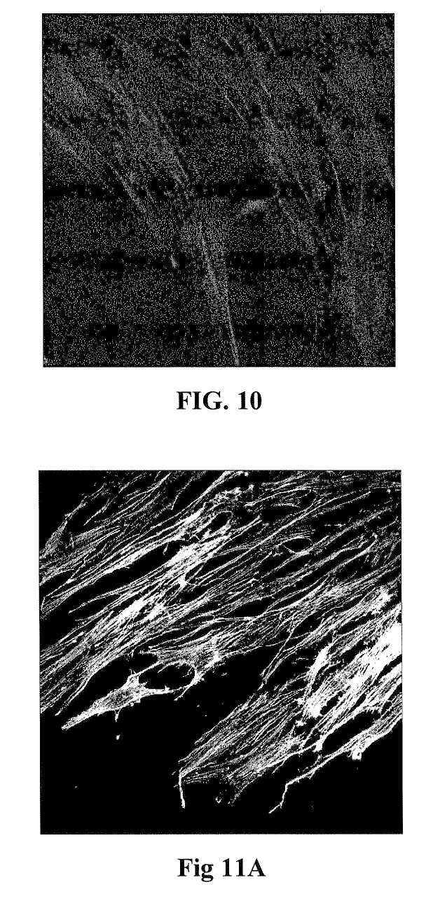

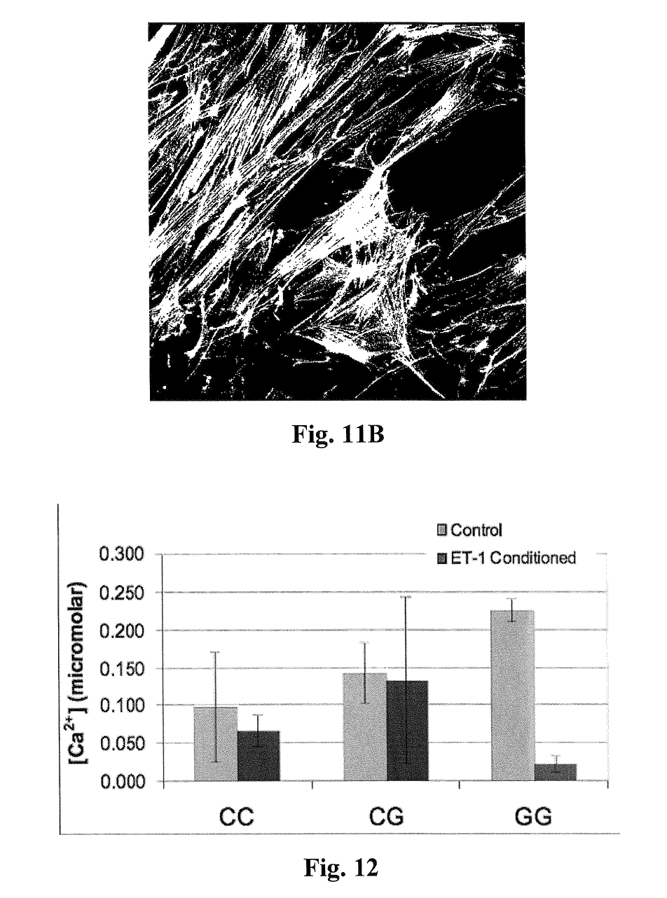

EDNRA is expressed on airway and vascular smooth muscle. Using indirect immunocytochemical staining of human ASM cells from the trachealis muscle retrieved from necroscopy tissue of 53 individuals, we have determined these cells consistently display EDNRA on the cell membrane (FIG. 10—staining is predominantly along cell membranes). Furthermore, preliminary data suggest that culturing the cells in the presence of endothelin-1 (ET-1) for 24 hours increases the formation of actin stress fibers (FIG. 11—TRITC-phalloidin shows increased formation of actin filaments implying functional EDNRA and expression of a more contractile phenotype). This implies a functional receptor with ligand binding and intact intracellular signaling pathways.

Smooth muscle cells of various EDNRA genotype combinations were examined for changes in intracellular calcium in response to endothelin. The effects of chronic exposure were assessed by incubating the cells for 24 hrs in endothelin, followed by a brief washout (30 minutes) and then re-exposure to endothelin and measurement of calcium release. Only cultures with the “protective” GG genotype showed downregulation of calcium release. For these assays, intracellular calcium mobilization from sarcoplasmic reticulum was measured using Fura-2. No significant differences were observed between subjects with regards to magnitude or duration of response from acute stimulation (FIG. 12, control). However, if cultured for 24 hrs in the presence of endothelin, washed out just before reexposure, there is a significant difference between subjects in the magnitude of response (FIG. 12, ET-1 conditioned). When compared by genotype, cells carrying the “protective” genotype of the 3′ markers showed substantial reductions in activity (on average, 15% of pre-exposure activity), while the “adverse” genotypes only lost about 10-15% activity.

Determination of EDNRA genotype may be advantageous in treatment of other diseases. Table 10 provides the results of a population study of a cohort of nearly 400 female CF patients divided into severe and mild lung disease categories, a cohort of 55 female Caucasian LAM patients and frequencies of the variants in individuals of African or European descent according to public databases for comparison. Frequencies (in percentages) for 5 markers in the EDNRA gene are provided. The first two columns of frequencies are alleles and the last three columns of frequencies are genotypes. At the bottom are putative haplotype constructions. For Caucasians and Africans, the haplotypes are based on the most common allele at each position. For the severe and mild, the haplotypes are in reference to each other and for LAM the haplotype is referenced to Caucasians. GMS refers to the CF Gene Modifier Study. As Table 10 shows, the distribution of EDNRA genotypes for 4 of the 5 variants in EDNRA show the same pattern in LAM patients as in severe CF patients, suggesting that LAM involves EDNRA activity.

TABLE 10

| SNP ID | rs#/Group | Chrom | BP Position | | | | | |
|---|---|---|---|---|---|---|---|---|
| 6023 | rs1429121 | 4 | 148708595 | A | C | AA | AC | CC |
| | GMS Severe | | | 55 | 45 | 29 | 51 | 20 |
| | GMS Mild | | | 65 | 35 | 47 | 34 | 18 |
| | Caucasians | | | 63 | 37 | 37 | 50 | 13 |
| | African Americans | | | 15 | 84 | 0 | 30 | 70 |
| | LAM | | | 45 | 55 | 10 | 70 | 20 |
| | All GMS fem | | | 61 | 39 | 41 | 40 | 19 |
| 6024 | rs4835405 | 4 | 148736289 | C | T | CC | CT | TT |
| | GMS Severe | | | 34 | 66 | 11 | 45 | 44 |
| | GMS Mild | | | 23 | 77 | 8 | 30 | 62 |
| | Caucasians | | | 33 | 67 | 10 | 45 | 45 |
| | African Americans | | | 72 | 28 | 44 | 56 | 0 |
| | LAM | | | 38 | 62 | 13 | 49 | 38 |
| | All GMS fem | | | 27 | 73 | 9 | 36 | 55 |
| 6016 | rs1801708 | 4 | 148759974 | A | G | AA | AG | GG |
| | GMS Severe | | | 42 | 58 | 18 | 48 | 34 |
| | GMS Mild | | | 27 | 63 | 9 | 35 | 56 |
| | Caucasians | | | 30 | 70 | 5 | 52 | 43 |
| | African Americans | | | 85 | 15 | 75 | 20 | 5 |
| | LAM | | | 40 | 60 | 15 | 49 | 36 |
| | All GMS fem | | | 32 | 68 | 12 | 40 | 48 |
| 6030 | rs10305895 | 4 | | A | G | AA | AG | GG |
| | GMS Severe | | | 76 | 24 | 56 | 40 | 4 |
| | GMS Mild | | | 78 | 22 | 60 | 34 | 6 |
| | Caucasians | | | 77 | 23 | 62 | 30 | 8 |
| | African Americans | | | 60 | 40 | 37 | 47 | 16 |
| | LAM | | | 76 | 24 | 52 | 48 | 0 |
| | All GMS fem | | | 76 | 24 | 57 | 38 | 5 |
| 6014 | rs5335 | 4 | 148821445 | C | G | CC | CG | GG |
| | GMS Severe | | | 50 | 50 | 21 | 59 | 20 |
| | GMS Mild | | | 36 | 64 | 14 | 45 | 41 |
| | Caucasians | | | 45 | 55 | 20 | 49 | 31 |
| | African Americans | | | 75 | 25 | 53 | 46 | 1 |
| | LAM | | | 43 | 57 | 20 | 46 | 34 |
| | All GMS fem | | | 41 | 59 | 16 | 50 | 34 |
| | All CF pts | | | 40 | 60 | 15 | 50 | 35 |

Haplotypes

| | | | | | |
|---|---|---|---|---|---|
| | 6023 | 6024 | 6016 | 6030 | 6014 |
| Af Am | C | C | A | A | C |
| Eur | A | T | G | A | G |
| Severe | C | C | A | G/A | C |
| Mild | A | T | G | G/A | G |
| LAM | C | C/T | A | A | G |

The data suggest that the adverse genotype combinations lead to greater production of EDNRA than the genotypes associated with mild disease in CF and LAM. The observation that these two variants show a stronger effect in combination than either SNP alone implies that either each region has an effect on function and that the two additive, or that other variants are involved and that these SNPs are markers for haplotypes underlying the effect.

In the lung, EDNRA acts to bind ET-1 to stimulate smooth muscle contraction, cell proliferation, and inflammation. Each of these events has been described as deleterious for the CF lung. ET-1, reported to be elevated in CF lungs, combined with an EDNRA genotype that promotes increased expression, predicts an environment that would be detrimental to the CF airway. The data indicate that mRNA levels are higher for those alleles associating with more severe disease.

In a separate screening, interleukin-8 (IL-8) was also shown to associate with CF severity in males. IL-8 is an integral component of CF lung disease; it is elevated, with substantial variation, in the lungs of CF patients.

An initial study cohort was made up of 737 CF patients enrolled in the Gene Modifier Study. All study subjects in cohort 1 are CF patients homozygous for the ΔF508 CFTR mutation. On the basis of the data obtained from the National CF Patient Registry, upper and lower quartiles of lung function for age were determined. Using these values as a guide, subjects were designated as ‘mild’ (n=491) or ‘severe’ (n=246), respectively, to reflect their lung function profile. A case-control study design was used to compare these groups, with patients with severe lung disease arbitrarily set as the case population and the mild group as the control population.

Genotyping was performed by SNP BeadArray (Illumina, Incorporated, San Diego, Calif., USA) technology and included the following polymorphic loci within IL8 (position relative to the transcriptional start site, number of patients genotyped): rs4073 (A-251T, n=733), rs2227307 (G396T, n=732) and rs2227543 (T1632C, n=737). Patients not genotyped by Illumina Inc. were genotyped at the following IL8 loci using restriction fragment length polymorphism (RFLP) assays. Primers and restriction enzymes used for the RFLP genotyping were (SNP, forward, reverse, product size, restriction endonuclease): rs4073, 5′-TGAAAAGTTGTAGTATGCCCC-3′ (SEQ. ID. NO. 3), 5′-CATTTAAAATACTGAAGCTCCACAATTTGGTGAATTATCgA-3′(SEQ. ID. NO. 4), 310 bp, ClaI; and rs2227543, 5′-TGGTTAAAAAAAAAGGAATAGCATCAATAGTGAGTTTGTTGTcCT-3′ (SEQ. ID. NO. 5), 5′-ACCCTACAACAGACCCACAC AATA-3′ (SEQ. ID. NO. 6), 420 bp, MnlI. Lowercase letters indicate a sequence change of the 3 SNP to introduce a restriction endonuclease recognition site. Digestion products were analyzed on a 2% agarose gel. Reactions were performed using Invitrogen Platinum Taq polymerase enzyme, 10× buffer, deoxyribonucleotides (2.5 mM) and MgCl2 (25 mM). Annealing temperature, MgCl2 concentration and amplification cycles were optimized for each primer set. Variants in the RASSF6 (rs13131954, n=717), IL8 (rs2227306, n=727), CXCL6 (rs4694639, n=709) and PF4VI (rs941758, n=716) genes were genotyped using an Assay-on-Demand assay (Applied Biosystems, Foster City, Calif., USA).

As Table 11 indicates, the evidence for association is more significant in the males than females, but is modest even in males. Therefore, an additional population was examined to determine if the association, particularly in males, could be replicated.

TABLE 11
Comparison of rs4073 genotypic P-values between
cohort 1 and 2 reveals gender effect

| | rs4073 | rs2227307 | rs2227306 | rs2227543 |
|---|---|---|---|---|
| Patient cohort 1 | | | | |
| Total (n = 737) | 0.07 | 0.04 | 0.19 | 0.06 |
| Males (n = 392) | 0.07 | 0.06 | 0.15 | 0.12 |
| Females (n = 345) | 0.25 | 0.12 | 0.36 | 0.19 |
| Patient cohort 2 | | | | |
| Total (n = 385) | 0.25 | 0.15 | 0.18 | 0.52 |
| Males (n = 219) | 0.01 | 0.08 | 0.01 | 0.06 |
| Females (n = 166) | 0.82 | 0.59 | 0.84 | 0.70 |

Three of the IL8 markers were typed in a second cohort. Patients in cohort 2 (n=385, 219 males) were ascertained by the same phenotypic criteria as cohort 1, but were not all homozygous for the ΔF508 mutation. They did, however, carry at least one copy of the ΔF508 CFTR mutation and another pancreatic insufficiency-associated mutation on the second allele. The same criteria for ‘mild’ and ‘severe’ used for cohort 1 were applied to the second cohort. Both cohorts were assessed for indices of pulmonary function, including average forced expiratory volume in 1 second (FEV₁), rate of decline in FEV₁ and predicted FEV₁ at age 20 (EBint20), as well as bacterial colonization and body mass index.

As Table 12 shows, the males in these two cohorts are not significantly different across indices of pulmonary function.

TABLE 12
Clinical characteristics of study cohorts

| | Severe males | | Mild males | |
|---|---|---|---|---|
| | Cohort 1 | Cohort 2 | Cohort 1 | Cohort 2 |
| Variable | (n = 130) | (n = 69) | (n = 303) | (n = 151) |
| Age-mean (±s.d.) (years) | 16.3 ± 4.5 | 17.1 ± 5.1 | 29.7 ± 9.9 | 31.5 ± 10.3 |
| Age-range (years) | 8.1-32 | 8-25.9 | 15.1-54.1 | 15.1-58.2 |
| FEV1 (±s.d.) | 47.0 ± 17.1 | 46.5 ± 17.8 | 69.8 ± 30.0 | 64.0 ± 29.9 |
| (% predicted) | | | | |
| FEV1 (±s.d.) | −3.59 ± 2.27 | −3.63 ± 2.01 | −1.41 ± 1.52 | −1.30 ± 1.59 |
| (decline %/year) | | | | |
| EBint20 (±s.d.) | 34.5 ± 17.4 | 35.0 ± 17.3 | 84.8 ± 15.3 | 82.0 ± 17.0 |
| Body mass index (±s.d.) | 17.5 ± 2.05 | 17.8 ± 2.80 | 22.4 ± 2.76 | 22.4 ± 2.78 |
| (percentile) | | | | |
| P. aeruginosa positive (%) | 85.4 | 85.7 | 85.8 | 90.4 |
| % DF508/ΔF508 | 100.0 | 63.0 | 100.0 | 66.7 |

Males and females in cohort 2 were assessed separately using a χ² test of association. In accordance with the first population, only male genotypes showed significant associations (0.01>P>0.08) in this second cohort (Table 4). The reproducibility of the association only in males indicates that gender is a major factor in IL8's role as a genetic modifier of CF. We also examined the IL8 SNPs for departures from Hardy-Weinberg proportions and found no evidence of such departures in either of the cohorts, nor in males alone (P>0.6).

The distribution of the marker alleles and genotypes in the severe group was compared to that in the mild group using a χ² test of independence. A goodness-of-fit test for Hardy-Weinberg Equilibrium (HWE) was performed by comparing the genotype frequencies with those expected on the basis of the observed allele frequencies. SNPs with a P<0.05 for the test of HWE were noted as possibly requiring careful consideration of the possible causes of departure from HWE, but were not removed from the analysis. The IL8 and flanking polymorphisms were found to exist in HWE, 0.27<P<0.96. All analyses were conducted in SPlus version X.0 (Insightful Corporation, Seattle, Wash., USA). The pattern of pairwise LD between SNPs is measured by the correlation coefficient (R²). R² varies from 0 to 1 and measures the correlation between alleles on the same chromosome. R² will take the value of 1 when the allele at one locus can always be predicted by the allele at the second locus (that is, if only two haplotypes are present).

The IL8 markers display high LD with each other and are therefore largely redundant in their information. An example of the genotype distribution in the studied subjects is provided for the promoter polymorphism A-251T (rs4073) in Table 13. The frequency of males homozygous for the A allele in the mild group is greater than in the severe group (25.6 vs 18.9%), while the frequency of T homozygotes is greater for the male subjects in the severe group (38.9 vs 25.1 %). Heterozygosity is found more often in the mildly affected males (49.3 vs 42.1%), suggesting a dominant or semi-dominant effect of the A allele on the mild phenotype. The SNP rs4073 is provided with flanking sequences as SEQ. ID. NO. 7.

TABLE 13
Comparison of rs4073 genotypes genotype frequencies
in cohorts 1, 2, and combined reveals gender effect

| | % AA | % AT | % TT | Genotypic P-value |
|---|---|---|---|---|
| Patient cohort 1 | | | | |
| All (n = 733) | | | | |
| Mild (n = 490) | 25.6 | 48.8 | 25.6 | 0.07 |
| Severe (n = 243) | 19.2 | 50.8 | 30.0 | |
| Males (n = 389) | | | | |
| Mild (n = 271) | 26.5 | 50.0 | 23.5 | 0.07 |
| Severe (n = 118) | 19.5 | 46.9 | 33.6 | |
| Patient cohort 2 | | | | |
| All (n = 379) | | | | |
| Mild (n = 242) | 22.7 | 46.7 | 30.6 | 0.25 |
| Severe (n = 137) | 20.4 | 42.3 | 37.2 | |
| Males (n = 219) | | | | |
| Mild (n = 149) | 23.6 | 47.9 | 28.6 | 0.01 |
| Severe (n = 70) | 17.5 | 33.3 | 49.2 | |
| Patient cohorts 1 and 2 | | | | |
| All (n = 1112) | | | | |
| Mild (n = 732) | 24.7 | 48.2 | 27.1 | 0.02 |
| Severe (n = 380) | 19.6 | 47.9 | 32.5 | |
| Males (n = 608) | | | | |
| Mild (n = 420) | 25.6 | 49.3 | 25.1 | 0.001 |
| Severe (n = 188) | 18.9 | 42.1 | 38.9 | |

A luciferase reporter assay was used to assess the IL8 promoter variant for effects at the level of transcription. IL8 promoter-luciferase constructs with either the A or T allele at variant rs4073 (rs4073A or rs4073T) were transiently transfected into 9HTEo-cells (pCEP-2), treated with a pro-inflammatory cytokine mix, and luciferase output measured at 6, 12 and 24 h post-treatment.

The IL8 promoter (1.5 kb) was cloned into a pGL3 vector (Promega Corporation, Madison, Wis., USA) containing the firefly luciferase gene. Using site-directed mutagenesis, two versions of the construct were generated to reflect the polymorphism at the −251 position (relative to the start of transcription), corresponding to SNP rs4073. The constructs are designated −251A/rs4073A and −251T/rs4073T.

These constructs were transiently co-transfected with the Renilla luciferase reporter construct (Promega) into control (pCEP-2) and CF-like (pCEP-RF) 9HTEo-cell lines using FuGene 6 Transfection Reagent (Roche Applied Science, Indianapolis, Ind., USA). As results from the pCEP-2 and pCEP-RF cells lines were similar, only data from pCEP-2 cells are shown.

To induce IL8-promoter driven luciferase expression, transfected cells were treated with physiologically relevant quantities of a pro-inflammatory cytokine mixture (interleukin-1β (0.167 ng/μl) and tumor necrosis factor-α (0.33 ng/μl) in serum-free culture media 24 h after transfection. Treatment with the cytokine mix was intended to mimic the pro-inflammatory state in CF airway in which there are increased levels of IL-1 β and TNF-α, both of which are considered key pro-inflammatory cytokines. Cells were also treated with hydrogen peroxide (H₂O₂), as oxidative stress has been shown to promote inflammation, which yielded a similar expression pattern to cytokine mix treatment (data not shown). Control cells were not exposed to stimulus, but rather to the phosphate-buffered saline diluent.

Cell lysates were harvested using the Dual-Luciferase Kit (Promega) at 6, 12, 24, 36 and 48, 60 and 72 h after treatment and luciferase activity measured in a luminometer (Turner BioSystems, Sunnyvale, Calif., USA). All samples returned to baseline by 24 h and thus data are shown for 6, 12 and 24 h time points only. The average relative luminescence was obtained for each condition (n=30 wells). Comparisons were made between treated and untreated cells, as well as between alleles. In all cases comparisons were assessed by a two-tailed, student's t-test.

The cytokine mix was found to significantly repress Renilla expression (P<0.04-P<0.005), thereby falsely inflating the magnitude of the observed response to the cytokine mix. Therefore, Renilla expression was only used to determine if transfection efficiencies were comparable for each time point. While expression estimates from the control samples were normalized to the co-transfected Renilla construct, the level of expression from the treated samples was not. Rather, multiple replicates were performed to ensure accuracy (30 individually transfected wells for each condition).

Treatment with the cytokine mix induced a significant increase in luciferase expression for both constructs (P<0.001), but under all conditions the T allele expressed 2-3 times more than the A allele (P<0.001). The fold induction of both constructs by the cytokine mix was similar and nonsignificant, however (P>0.56, data not shown).

### Materials and Methods

Statistical Analysis: Single-marker tests of association were conducted using χ² goodness of fit test and comparing to the method of genomic control to account for the possibility of spurious associations because of undetected population stratification within Caucasian populations. The genomic control parameter, λ, was calculated from 300 markers typed in this cohort. The test statistic for each marker is adjusted by dividing by λ, and the p-value is obtained from the χ² distribution using this adjusted test statistic. If population stratification is present, the test statistics will be inflated for many markers, and the distribution of the test statistic will be shifted. The value of λ was found to be 1.0 for females and 1.14 for males (where 1.0 represents no evidence of population stratification). Of the markers examined, only SNPs in or near EDNRA did not exceed the threshold of p=0.05.

Based upon the foregoing disclosure, it should now be apparent that the invention disclosed herein will carry out the objects set forth hereinabove. It is, therefore, to be understood that any variations evident fall within the scope of the claimed invention and thus, the selection of specific component elements can be determined without departing from the spirit of the invention herein disclosed and described.

We claim:

1. A method of determining a genetic component contributing to the severity of a pulmonary disease in a patient, the method comprising determining the genotype (sequence) of one or more single nucleotide polymorphisms (SNPs) in the Endothelin Receptor A (EDNRA) gene or the Interleukin-8 (IL-8) gene of the patient, wherein the SNPs are selected from group consisting of rs5335 and rs1801708 for EDNRA and rs 4073 for IL-8.

2. The method of claim 1, wherein the pulmonary disease is selected from the group consisting of cystic fibrosis and lymphangioleimyomatosis.

3. The method of claim 2, wherein rs5335 comprises a guanine (G) or a cytosine (C) nucleotide corresponding to position 239 of SEQ. ID. NO. 1.

4. The method of claim 2, wherein rs1801708 comprises a guanine (G) or adenine (A) nucleotide corresponding to position 301 of SEQ. ID. NO. 2.

5. The method of claim 2, wherein rs 4073 comprises an adenine (A) or a thymidine (T) nucleotide corresponding to position 301 of SEQ. ID. NO. 7.

6. A method of treating a patient having a pulmonary disease, the method comprising determining the genotype (sequence) of one or more single nucleotide polymorphisms (SNPs) in the Endothelin Receptor A (EDNRA) gene or the Interleukin-8 (IL-8) gene of the patient, wherein the SNPs are selected from group consisting of rs5335 and rs1801708 for EDNRA and rs 4073 for IL-8.

7. The method of claim 6, wherein the pulmonary disease is selected from the group consisting of cystic fibrosis and lymphangioleimyomatosis.

8. The method of claim 6, wherein rs5335 comprises a guanine (G) or a cytosine (C) nucleotide corresponding to position 239 of SEQ. ID. NO. 1.

9. The method of claim 6, wherein rs1801708 comprises a guanine (G) or adenine (A) nucleotide corresponding to position 301 of SEQ. ID. NO. 2.

10. The method of claim 6, wherein rs 4073 comprises an adenine (A) or a thymidine (T) nucleotide corresponding to position 301 of SEQ. ID. NO. 7.

11. A kit for performing the method of claim 1, the kit comprising one or more probes for determining the determining the genotype (sequence) determining the genotype (sequence) of one or more of the single nucleotide polymorphisms (SNPs) rs5335 and rs1801708 in the Endothelin Receptor A (EDNRA) gene or the rs 4073 SNP for the Interleukin-8 (IL-8) gene of the patient.

12. The kit of claim 11, wherein rs5335 comprises a guanine (G) or a cytosine (C) nucleotide corresponding to position 239 of SEQ. ID. NO. 1.

13. The kit of claim 11, wherein rs1801708 comprises a guanine (G) or adenine (A) nucleotide corresponding to position 301 of SEQ. ID. NO. 2.

14. The kit of claim 11, wherein rs4073 comprises an adenine (A) or a thymidine (T) nucleotide corresponding to position 301 of SEQ. ID. NO. 7.

15. The kit of claim 11, wherein the probe is a nucleic acid comprising at least residues 221-239 of SEQ. ID. NO. 1 or the reverse complement thereof.

16. The kit of claim 11, wherein the probe is a nucleic acid comprising at least residues 239-250 of SEQ. ID. NO. 1 or the reverse complement thereof.

17. The kit of claim 11, wherein the probe is a nucleic acid comprising at least residues 290-301 of SEQ. ID. NO. 2 or the reverse complement thereof.

18. The kit of claim 11, wherein the probe is a nucleic acid comprising at least residues 301-320 of SEQ. ID. NO. 2 or the reverse complement thereof.

19. The kit of claim 12, wherein the probe is a nucleic acid comprising at least residues 283-301 of SEQ. ID. NO. 7 or the reverse complement thereof.

20. The kit of claim 12, wherein the probe is a nucleic acid comprising at least residues 301-319 of SEQ. ID. NO. 7 or the reverse complement thereof.
